(19) United States
(12) Patent Application Publication
(10) Pub. No.: US 2025/0281750 A1
(43) Pub. Date: Sep. 11, 2025

(54) IMPLANTABLE ELECTROCORTICOGRAM BRAIN-COMPUTER INTERFACE SYSTEMS FOR MOVEMENT AND SENSATION RESTORATION

(71) Applicants: THE REGENTS OF THE UNIVERSITY OF CALIFORNIA, Oakland, CA (US); UNIVERSITY OF SOUTHERN CALIFORNIA, Los Angeles, CA (US); CALIFORNIA INSTITUTE OF TECHNOLOGY, Pasadena, CA (US)

(72) Inventors: An H. Do, Irvine, CA (US); Payam Heydari, Irvine, CA (US); Zoran Nenadic, Irvine, CA (US); Charles Liu, Los Angeles, CA (US); Richard Andersen, Pasadena, CA (US)

(21) Appl. No.: 18/862,752

(22) PCT Filed: Jun. 20, 2023
(86) PCT No.: PCT/US23/68700

§ 371 Date: Nov. 4, 2024

Related U.S. Application Data
(60) Provisional application No. 63/353,812, filed on Jun. 20, 2022.

(51) Int. Cl. A61N 1/36; A61N 1/05; A61N 1/372; A61N 1/378

(52) U.S. Cl. CPC A61N 1/36103; A61N 1/0531; A61N 1/36125; A61N 1/36139; A61N 1/37217; A61N 1/3787

(57) ABSTRACT

An implantable medical device to restore brain-controlled movement and sensation after neural injury. The device comprises a brain-computer interface capable of acquiring electrocorticogram (ECoG) signals recorded directly from the surface of the brain and uses the signals to enable direct control of paralyzed muscles, limbs or extremities while simultaneously receiving signals from external sensors and converting them into electrical stimulation patterns for the brain's sensory areas.

### CROSS-REFERENCES TO RELATED APPLICATIONS

This application claims benefit of U.S. Provisional Application No. 63/353,812 filed Jun. 20, 2022, the specification of which is incorporated herein in its entirety by reference.

### STATEMENT REGARDING FEDERALLY SPONSORED RESEARCH OR DEVELOPMENT

This invention was made with government support under Grant No. 1646275 awarded by the National Science Foundation and Grant No. CNS1646307 awarded by the National Science Foundation. The government has certain rights in the invention.

### FIELD OF THE INVENTION

The present invention relates to systems and devices which interface between a brain and a computer. More specifically, the present invention relates to implantable systems and devices to provide for brain-controlled walking and sensation.

### BACKGROUND OF THE INVENTION

Clinicians have traditionally relied on physiotherapies and assistive devices, such as orthoses, wheelchairs, and functional electrical stimulation (FES) systems, to mitigate motor impairments after spinal cord injury (SCI). However, conventional physiotherapies provide only gains and functional recovery typically plateaus 6 months after injury. Moreover, assistive devices, such as RGEs, only partially address the functional impairments and their benefits disappear upon removal. In addition, they are often perceived as cumbersome and uncomfortable, and they typically require manual control, thereby monopolizing the use of residual motor functions. Furthermore, none of these approaches can restore sensory functions. Emerging techniques, such as cellular therapies or neuromodulatory approaches, may be viable strategies, but are in very early phases of investigation and early data indicates that they may only be useful in helping those with incomplete SCI.

Therefore, the loss of walking and sensory functions after SCI has been suboptimally addressed, and there is a desperate need for effective solutions to these problems. Such a solution would improve quality of life, increase independence, and reduce the incidence of medical complications in people with SCI, which in turn would reduce the associated healthcare and caregiving costs.

Previous solutions also include brain-computer interface (BCls) systems that restore only walking. Electroencephalogram (EEG) based BCI could restore basic treadmill and overground walking to a person with paraplegia due to SCI. Despite these encouraging results, widespread adoption of EEG-based BCI prostheses in the future is unlikely. This is because of EEG's limited spatiotemporal resolution (>1 cm, <35 Hz) and susceptibility to motion artifacts that may lead to erroneous BCI control, causing user frustration and/or injury. Also, except for visual feedback, EEG-based BCIs lack sensory information. Finally, EEG cap mounting and dismounting procedures are tedious and time-consuming, and EEG equipment is generally seen as cumbersome and aesthetically unpleasing to users. While progress has been made in EEG electrode technology, the above limitations remain largely unsolved.

Brain-computer interfaces (BCIs) allow users to directly translate their motor intention measured from electrophysiological or other signals of the brain to control external devices to carry out desired actions. The advancement in electrophysiological signal acquisition and decoding has demonstrated promising results in motor control of robotic limbs or muscle stimulation through one-way communication between the brain and external devices. In BCI applications where no-feedback other than visual feedback is necessary such as keyboard typing, open-loop, uni-directional BCI may be sufficient. However, real-life movement invariably involves continuous interaction with external objects and environments such as in the case of grasping a delicate object. In human motor control, the role of sensory feedback in movement planning, control, and motor learning is known to play an integral part in necessitating complex sensorimotor integration. The theory of optimal feedback control affirms that humans rely on cost and rewards, internal models, optimal feedback-driven policy, and state estimation, all of which demand somatosensory feedback as a crucial component of normal motor control. Physiological studies corroborate that the loss of somatosensation causes severe deficits in motor control. Therefore, an important challenge for BCI development has been to create technologies that can convey sensory information simultaneously with motor decoding, to realize a bidirectional BCI (BD-BCI) system.

BD-BCI progress largely focused on characterizing the sensory stimulation in evoking sensation, which requires an artificial sensory stimulator. Recently, a closed-loop BD-BCI demonstrated improved prosthetic arm motor control. However, operations of the existing BD-BCI systems are limited to a laboratory setting where the systems run only on bulky non-mobile workstation computers, data acquisition systems, and commercial stimulators. For BD-BCIs to become practical, all of the above components must be integrated into a special purpose and compact form factor with full programmability. Most importantly, it must be shown to be safe-specifically equivalent to predicate FDA-approved cortical stimulators. The present invention features a stimulator system integrated with an embedded BCI system with rigorous comparison against an FDA-approved cortical stimulator as a critical step towards this goal.

Electrocorticogram (ECoG) based-BCIs and intracortical microelectrode-based BCIs for restoring walking also only claim to provide motor restoration. Furthermore, neuromodulation approaches such as spinal cord stimulators have not yet demonstrated that they can work across the entire spectrum of neurological injuries

### BRIEF SUMMARY OF THE INVENTION

It is an objective of the present invention to provide systems devices and methods that allow for brain-controlled limb movement and sensation, as specified in the independent claims. Embodiments of the invention are given in the dependent claims. Embodiments of the present invention can be freely combined if they are not mutually exclusive.

BCIs are cyber-physical systems (CPSs) that record human brain waves and translate them into the control commands for external devices such as computers and robots. They may allow individuals with SCI to assume direct brain control of an extremity prosthesis to regain the ability to walk. Since extremity paralysis due to SCI leads to as much as $50 billion of health care costs each year in the US alone, the use of a BCI-controlled extremity prosthesis to restore limb movement can have a significant public health impact.

Recent results have demonstrated that a person with paraplegia due to SCI can use a non-invasive BCI to regain basic walking. Results have also demonstrated that it is possible to deliver electrical stimulation to the brain to elicit artificial sensation, thereby providing a means to restore sensation to persons with paraplegia due to SCI. In addition, methods have been developed to eliminate the electrical artifact due to stimulation to maintain the integrity of the brain signal recorded in adjacent brain areas.

The envisioned grand scheme of the fully-implantable BD-BCI system proposes a hypothetical scenario where a person with SCI is implanted with the skull unit (SU) and the chest wall unit (CWU) connected by a tunneling cable subcutaneously (FIG. 1). The ECoG electrodes are implanted over the sensorimotor cortex and the downstream motor signal from motor cortex is amplified, multiplexed, and digitized in the SU which is then decoded in the CWU. The decoded motor commands are wirelessly transmitted to the limb prosthesis to actuate movement. Sensors within the prosthesis encoded limb kinematics and are sent wirelessly back to the CWU, where the encoded sensory information will be converted into electrical stimulation patterns. The electrical stimulation will be delivered to the sensory brain via the tunneling cable, multiplexed in the SU to target specific loci, thereby eliciting an artificial limb sensation.

The invention features a fully implantable BCI capable of acquiring electrocorticogram (ECoG) signals, recorded directly from the surface of the brain, and analyzing them internally to enable direct brain control of an external prosthetic system, including robotic gait exoskeleton (RGE) for walking, upper extremity prosthesis, upper extremity robotic exoskeleton, functional electrical stimulation system of the upper and/or lower extremities, or spinal cord stimulator for upper/lower extremities. The system takes signals from external sensors and converts them into electrical stimulation patterns for the brain's sensory areas. Artifact rejection mechanisms may be used to maintain the integrity of the ECoG signals so that they can simultaneously be used for accurate decoding of the user's movement intentions.

One of the unique and inventive technical features of the present invention is that ECoG grids are implanted over both the left and right primary motor cortex (M1) limb area and the left and right primary sensory cortex (S1) limb area. Without wishing to limit the invention to any theory or mechanism, it is believed that the technical feature of the present invention advantageously provides for simultaneous control of a prosthesis system and also artificial sensation from the prosthesis system. None of the presently known prior references or work has the unique inventive technical feature of the present invention. Furthermore, the prior references teach away from the present invention. For example, the prior art teaches that simultaneous stimulation and monitoring of brain wave signals can result in the introduction of artifacts in the recorded data.

Studies have used electrical stimulation of the spinal cord to generate walking, but do not necessarily provide any improvement in sensation for those with the most severe neurological injuries. The present invention provides a means to restore sensation. Additionally, previous studies have only employed uni-directional brain-machine interfaces, while the present invention provides sensory feedback to enable the user to have more accurate and safe control of the system.

Previous studies have proposed delivering electrical stimulation via microelectrodes placed intracortically. The present invention is less invasive and uses ECoG electrodes placed on the surface of the brain within the subdural space. In addition, this also results in longer viability of the electrodes and more stability of the electrical stimulation parameters as scarring is not as extensive. Previous studies have also proposed utilizing intracranial electronics to generate the stimulation pulses. The present invention instead generates electrical impulses for sensory stimulation from an implanted pulse generator (which also houses the BCI electronics) placed within the chest. This minimizes the potential exposure of the brain and surrounding tissue to the heat generated by the system.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows an illustration of the prosthesis component, the implantable BCI component, the tunneling cable, and the chest wall unit, in use by a patient.

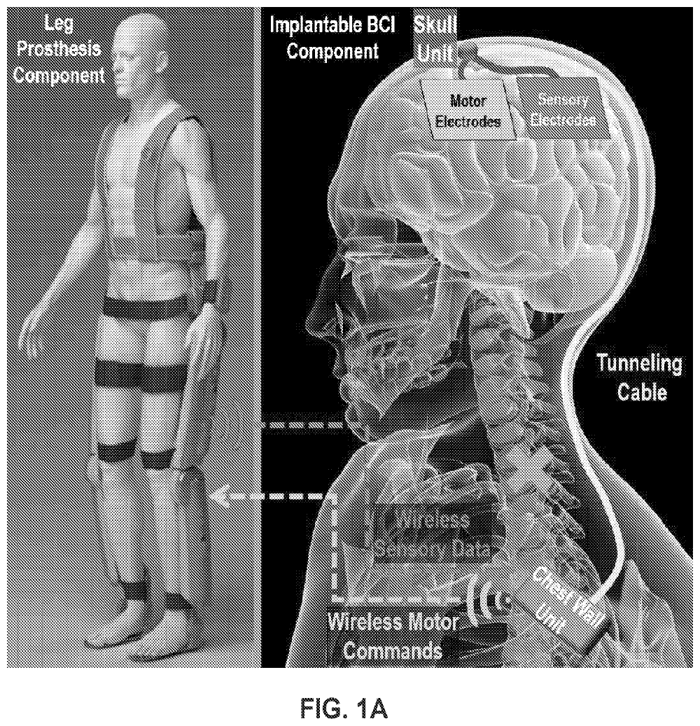

FIG. 1B shows a schematic of the implantable BCI component of the present invention.

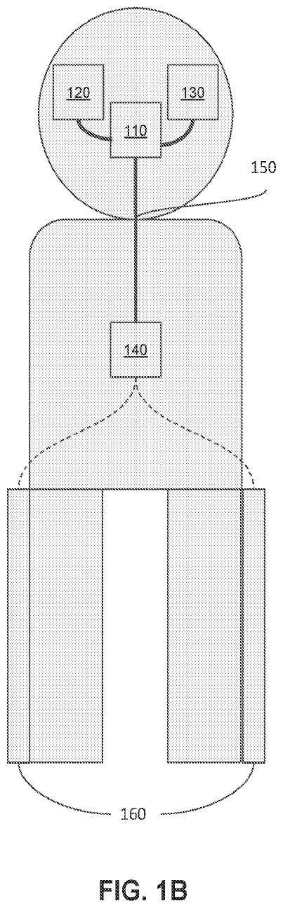

FIG. 1C shows a flow chart for a method for implanting the BCI component of the present invention.

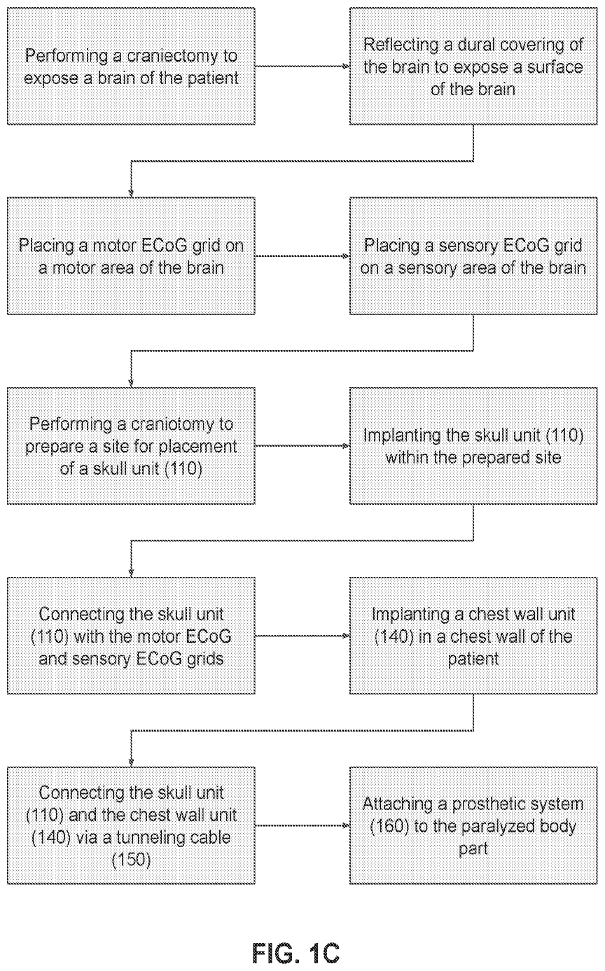

FIG. 2 shows a system block diagram of the system of the present invention.

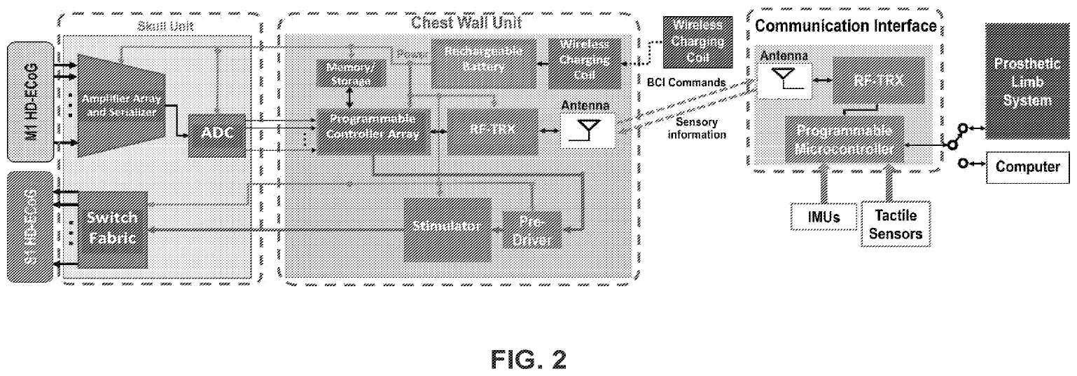

FIG. 3 shows a series of diagrams and photographs illustrating the implantation of the components of the present invention.

FIG. 4 shows a diagram of the operation of the prototype implantable BD-BCI stimulator. Compressing the structure of the envisioned fully implantable BCI system on a custom-printed circuit board, the CWU analog supplies electrical pulse trains to the SU analog which has a connector interfaced with ECoG electrodes. The CWU, composed of a multi-core microcontroller (MCU) cluster and supporting components, performs all necessary processing to control electrical stimulation. The base station is used to wirelessly configure the implantable BD-BCI stimulator through a medical (ISM) radio band. The BD-BCI stimulator is powered by a rechargeable battery that can be charged wirelessly.

FIG. 5 shows the design schematic of the stimulator. The CWU comprises a microcontroller, H-bridge, a current source (I-src), a charge pump, LDO, digital rheostats, a current sensor, and a battery. SU analog comprises the three multiplexers (A1-A3) and charge-monitor. ECoG electrodes are plugged into standard touch-proof jacks. F1, F2, F3: feedback signals for voltage and impedance monitoring. Isense: current sensor. InAmp: Instrumentation amplifier. PULSE GEN: pulse generator. REF: reference electrode.

FIGS. 6A-6C show schematic embodiments and results of the present invention. FIG. 6A shows a schematic of the circuit and the ECoG grid in phantom brain tissue. FIG. 6B shows an illustration of a biphasic pulse and the voltage sampling timing. FIG. 6C shows a sample state machine implementing a threshold-based active charge balancing. tₐ: anodic pulse width. t_{c}: cathodic pulse width. t₁: sampling timing for impedance measurement. t₂: sampling timing for active charge balancing (steady state voltage). V_{H}: upper voltage threshold. V_{L}: lower voltage threshold.

### DETAILED DESCRIPTION OF THE INVENTION

The present invention features a fully-implantable brain-computer interface system (100) comprising a skull unit (110), implanted in the skull of a patient, a motor grid (120), comprising electrocorticogram (ECoG) motor electrodes implanted in a subdural space over a motor area of a brain of the patient, configured to detect a brain wave motor signal, the motor grid (120) connected to the skull unit (110) via a wire, a sensory grid (130), comprising ECoG sensory electrodes implanted in a subdural space over a sensory area of the brain of the patient, configured to provide an electrical stimulation to the sensory area, the sensory grid (130) connected to the skull unit (110) via a wire, a chest wall unit (140) having wireless communication capabilities, implanted within the chest wall of the patient, a subcutaneous tunneling cable (150) connecting the skull unit (110) and the chest wall unit (140), and an external prosthetic system (160) comprising a sensor, configured for wireless communication with the chest wall unit (140). The system (100) may be configured to control the prosthetic system (160) based on the brain wave motor signal, and the system (100) may be configured to provide the electrical stimulation to the sensory area based on activation of the sensor, thereby providing an artificial sensation to the user.

The present invention features a fully implantable brain-computer interface (BCI) to allow for simultaneous control of a body part of a patient and sensation from the body part. The BCI may comprise a skull unit (110) implanted within a skull of the patient, a motor grid (120), comprising electrocorticogram (ECoG) motor electrodes implanted in a subdural space over a motor area of a brain of the patient, configured to detect a brain wave motor signal, the motor grid (120) connected to the skull unit (110) via a wire, and a sensory grid (130), comprising ECoG sensory electrodes implanted in a subdural space over a sensory area of the brain, configured to provide an electrical stimulation to the sensory area, the sensory grid (130) connected to the skull unit (110) via a wire. The skull unit (110) may be configured to allow for control of the body part using the brain wave motor signal and sensation from the body part using the electrical stimulation to the sensory area.

The present invention features a method of restoring movement and sensation of a paralyzed body part of a patient. The method may comprise performing a craniectomy to expose a brain of the patient, reflecting a dural covering of the brain to expose a surface of the brain, placing a motor electrocorticogram (ECoG) grid on a motor area of the brain. The motor ECoG grid may be configured to detect a brain wave motor signal. The method may further comprise placing a sensory ECoG grid on a sensory area of the brain. The sensory ECoG grid may be configured to provide an electrical stimulation to the sensory area. The method may further comprise performing a craniotomy to prepare a site for placement of a skull unit (110), implanting the skull unit (110) within the prepared site, connecting the skull unit (110) with the motor ECoG and sensory ECoG grids, implanting a chest wall unit (140) in a chest wall of the patient, connecting the skull unit (110) and the chest wall unit (140) via a tunneling cable (150), and attaching a prosthetic system (160) to the paralyzed body part. The prosthetic system (160) may be configured for wireless communication with the chest wall unit (140). The prosthetic system (160) may comprise a sensor. Communication of the brain wave motor signal to the prosthetic system (160) restores movement of the paralyzed body part, and communication of a status of the sensor to the sensory area via the electrical stimulation of the area restores sensation of the paralyzed body part.

The external prosthetic system (160) may comprise a robotic gait exoskeleton (RGE), upper extremity prosthesis, upper extremity robotic exoskeleton, functional electrical stimulation system of the upper or lower extremities, or spinal cord stimulator for the upper or lower extremities. The motor area of the brain of the patient may comprise a left primary motor cortex limb area and a right primary motor cortex limb area. The sensory area of the brain may comprise a left primary sensory cortex limb area and a right primary motor cortex limb area. The skull unit (110) may comprise an amplifier array, an analog-to-digital converter, a stimulator multiplexor array, or a combination thereof. The chest wall unit (140) may comprise a a multi-core microcontroller (MCU) cluster, a memory/storage component, and a radio transceiver. The system (100) may further comprise a rechargeable battery configured to recharge wirelessly.

In the present invention, brain signals underlying movement intentions are sensed by motor electrodes, amplified and serialized into a single path by a skull unit (SU), and routed out of the head and neck using a subcutaneous tunneling cable. The signals are decoded using BCI algorithms executed on an embedded system housed within a chest wall unit (CWU). The CWU wirelessly transmits commands to a prosthesis to actuate movements. The end effector may include options such as robotic gait exoskeleton (RGE) for walking, upper extremity prosthesis, upper extremity robotic exoskeleton, functional electrical stimulation system of the upper and/or lower extremities, or spinal cord stimulator for upper/lower extremities. Sensors worn on the end effector measure movements and tactile information and send signals wirelessly back to the CWU, where they are converted into electrical stimulation patterns. These are delivered to the brain via the tunneling cable and sensory electrodes, thereby eliciting artificial limb sensation.

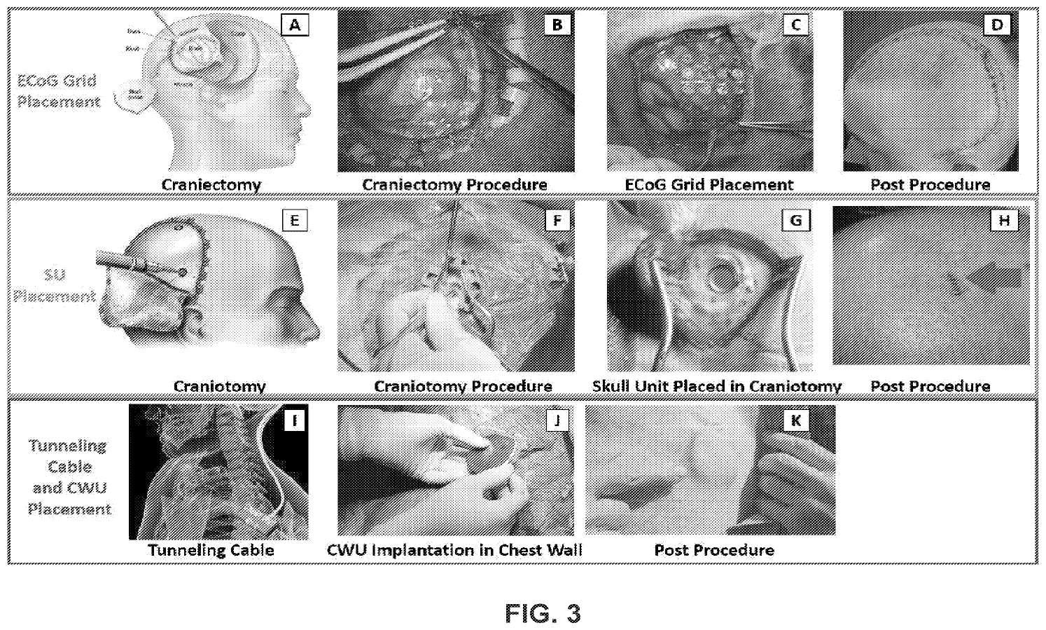

The system's fully implantable nature leaves no components protruding out of the body, thereby making the system socially and aesthetically acceptable to the potential users. Referring now to FIG. 3, the following surgical procedure may be used to implant the system. An ECoG grid will be implanted over each of the following brain areas: left and right primary motor cortex (M1) limb area, left and right primary sensory cortex (S1) limb area (total of 4 grids). This is achieved by performing a craniectomy (removal of the skull) over the midline areas of the head (A-B). The dural covering of the brain is reflected, and the ECoG grids will be placed over the areas listed above (C). The dura is placed back and the ECoG grids are sutured to the dura for fixation. The skull is then placed back (D). The cables from the ECoG grids are tunneled through the defect at the skull craniectomy site. Nearby, a craniotomy (creating a small hole in the skull, E-F) is performed so as to embed the skull unit (G). The cables from the ECoG electrodes are connected to the skull unit. The tunneling cable is also connected to the skull unit. Using a tunneling tool, this cable is “snaked” underneath the scalp and skin of the neck (I) to the chest (pectoral) area. An incision is made at the pectoral area and the exposed tunneling cable is connected to the CWU (J). The CWU is then placed into a subcutaneous cavity created at the incision site. All surgical wounds at the scalp and pectoral areas are then closed with sutures and surgical staples (D, H, K).

ECoG signals are sensed by the HD-ECoG grids implanted bilaterally in the relevant primary motor cortex of the brain. These signals are then fed to one of two selectable pathways. During online BCI operation, the DSP decodes ECoG signals to generate real-time BCI commands, which are sent wirelessly to a prosthesis system via the bi-directional custom communication interface (CCI). The prosthesis system may include robotic exoskeletons, functional electrical stimulation systems, or spinal cord stimulator systems. This CCI also sends gyroscope and foot pressure sensor signals from the RGE back to the CWU analog. The DSP converts the sensor data into a gating signal which activates the stimulator. The stimulator delivers stimulation pulses to selected channels within the HD-ECoG grids implanted in the relevant primary sensory cortex of the brain. Note that these grid sizes are constrained by the brain anatomy. The channel selection is performed by the artificial sensation algorithm. The switch fabric then toggles the selected channels.

Setting up and configuring the BD-BCI implant system is facilitated by a PC-based base station program with a graphical user interface (GUI) that is used to communicate with the CCI and subsequently to any BD-BCI implant. This is established via wireless communication which complies with the FCC MedRadio standard. The GUI can be used to program or reprogram the DSP by uploading a new firmware image and transmitting it to the BD-BCI via the CCI. Likewise, the GUI can be used to program all the settings of the BD-BCI. The GUI is also used to interact with the BD-BCI to initiate critical functions, the learning mode, and the online mode. The GUI is also used to download any data on the BD-BCI to a computer for external storage or telemetry.

The GUI itself can be placed into either a “Developer/Debug mode” or an “Operators Mode.” In the Developer/Debug mode, all the functions of the BD-BCI may be accessed via command lines. This is intended for developers, researchers, and other appropriately trained professionals to control a BD-BCI implant. The Operator mode provides access to the basic functions that allow an operator, such as a trained physician or other health care provider, to control a BD-BCI implant.

The operator of the base station program can establish a wireless connection between the CCI and a BD-BCI implant within about 10-30 ft. If the operator needs to recharge the battery in the BD-BCI, the CCI can be placed over the pectoral area where the BD-BCI implant resides in the user. This enables the wireless charging coil inside the CCI to induct the counterpart receiving coil in the BD-BCI to charge its battery.

The BD-BCI system can operate in 2 modes, a “learning mode” and an “online mode.” The Learning Mode has 2 sub-functions, which include motor learning mode and sensory learning mode. In the motor learning mode, ECoG data is acquired underlying the relevant attempted motor behavior. A decoding algorithm executed on the CWU analyzes the data to generate a decoding model, which will later be used to analyze ECoG data for real-time operation. In the sensory subfunction, the operator can choose pairs of ECoG electrodes to stimulate (including selecting stimulation parameters) to identify which electrode pair and stimulation parameters provide the optimal desired artificial sensation. The parameters can be set for future use in the online mode of operation.

In the online mode, ECoG data is acquired in real-time, decoded and commands are sent to the end effector. Sensors that detect the position of the end effectors and tactile information will be fed back to the BD-BCI system. Using the parameters defined in the learning mode, electrical stimulation will be delivered to elicit artificial sensations accordingly. It should be noted that the GUI will also be used to set up the BD-BCI's wireless connection to the external end effector and sensory feedback systems.

The CCI will also be used to program or reprogram the BD-BCI system and to transmit raw ECoG data to a computer for external storage or telemetry. To reprogram the BD-BCI, the firmware image will be compiled on an external computer. Using the GUI, the image will be uploaded onto the BD-BCI's storage space by wireless connection via the CCI. A built-in software/hardware mechanism will restart and flash the BD-BCI with the new firmware image. To monitor the ECoG from the brain in real-time, the BD-BCI can be commanded to transmit the signals wirelessly to the CCI, and subsequently, the signals will be rendered and displayed on the GUI for inspection.

The present invention features a fully-implantable brain-computer interface system (100). In some embodiments, the system (100) may comprise a motor grid (120), comprising electrocorticogram (ECoG) motor electrodes, configured to detect a brain wave motor signal. The system (100) may further comprise a first unit (110) operatively connected to the motor grid (120) via a first wire. The first unit (110) may be configured to amplify and serialize the brain wave motor signal into a single path. The system (100) may further comprise a sensory grid (130), comprising ECoG sensory electrodes, configured to provide an electrical stimulation. The sensory grid (130) may be operatively connected to the first unit (110) via a second wire. The system (100) may further comprise a second unit (140) configured to have wireless communication capabilities, a cable (150) configured to connect the first unit (110) and the second unit (140), and an external prosthetic system (160) comprising a sensor, configured for wireless communication with the second unit (140). The system (100) may be configured to control the prosthetic system (160) based on the brain wave motor signal and may be configured to provide the electrical stimulation based on the activation of the sensor.

In some embodiments, the external prosthetic system (160) may comprise a robotic gait exoskeleton (RGE), upper extremity prosthesis, upper extremity robotic exoskeleton, or functional electrical stimulation system. In some embodiments, the first unit (110) may comprise an amplifier area, an analog-to-digital converter, a stimulator multiplexor array, or a combination thereof. In some embodiments, the second unit (140) may comprise a multi-core microcontroller (MCU) cluster, a memory and storage component, and a radio transceiver. In some embodiments, the system (100) may further comprise a rechargeable battery configured to recharge wirelessly.

### EXAMPLE

The following is a non-limiting example of the present invention. It is to be understood that said example is not intended to be limiting in any way. Equivalents or substitutes are within the scope of the present invention.

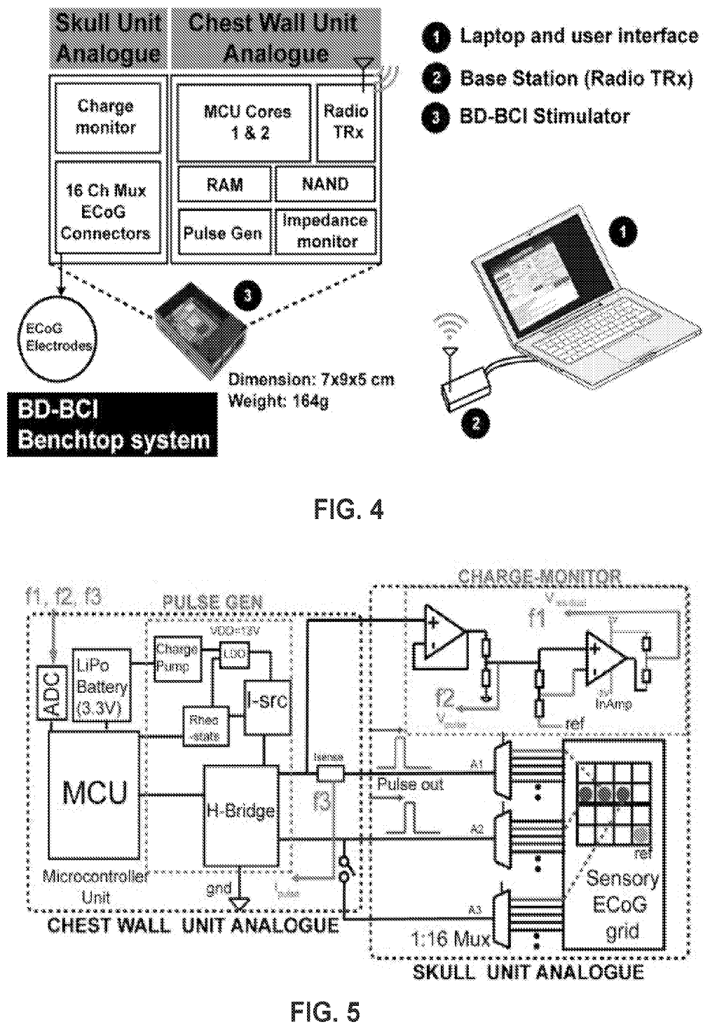

The electrical stimulator (BD-BCI stimulator) is integrated into a miniaturized benchtop fully-implantable BCI system. The CWU and SU analog are modularized and custom-designed on the printed circuit board (PCB) (FIG. 4). The CWU analog supplies electrical pulses to the SU analog which is interfaced with ECoG electrode grids. The CWU is composed of dual microcontroller cores (two 48 MHz ARM® Cortex-M0+ microcontrollers; Microchip, Chandler, AZ) and supporting components to maximize the resource and interfacing with peripherals such as 32-channel commercial bioamplifier integrated circuit with an integrated multiplexer and 16-bit analog-to-digital converter (ADC) (Intan Technology®, Santa Monica, CA), MedRadio band radio transceiver (TRX) (HOPE Microelectronics®, Xili, Shenzhen, China), memory (two 512-KiB FRAM; Cypress Semiconductor®, San Jose, CA), and storage modules (512-MiB NAND flash memory; Micron®, Boise, ID). The wireless control and data transmission between the base station and the TRX in the CWU (FIG. 4) is designed to comply with Federal Communication Commission designated Medical Device Radiocommunications Service (FCC MedRadio) for implantable medical devices. The benchtop BD-BCI system is battery-powered and can be charged wirelessly (Qi® 2.1 standard).

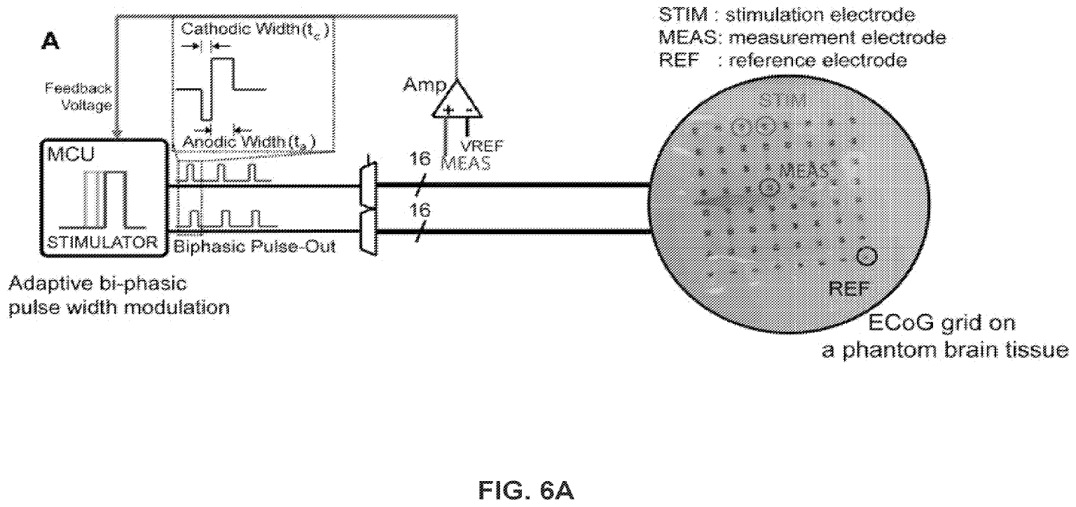

The stimulator was designed to be controlled by one of the microcontroller (MCU) cores of the previously developed benchtop system for a fully-implantable BCI interface. Biphasic square pulses were generated by a digitally controlled H-bridge (Texas Instrument®, Dallas, TX). The H-bridge was driven by a current source (Linear Technology®, Norwood, MA), two cascaded charge pumps (Maxim Integrated®, San Jose, CA), and an LDO (Texas Instrument®, Dallas, TX), which collectively boost voltage from 3.3V (Vcc) to 13V to meet the current demand (FIG. 5). The current level was digitally controlled by adjusting input parameters for the current source. A pair of 1:16 multiplexers (A1-A2) (Analog Devices®, Norwood, MA) enabled a selection of the electrode pair for a bipolar stimulation from a pool of up to 16 ECoG electrodes. A third 1:16 multiplexer (A3) enabled an extra electrode to be optionally selected to form a triple-pole artifact mitigation path. The microcontroller Timer/Counter for Control Applications (TCC) peripheral was used to trigger stimulation with precise digital control of the pulse duration, frequency of stimulation, and train duration. A breakout-board-interface with industrial standard 1.5 mm touch-proof connectors was designed to facilitate connection from the BD-BCI prototype stimulator and amplifier array to ECoG electrodes and accommodates up to 16 stimulation electrodes and 16 recording channels, reference, and the ground (GND) connection. A kill-switch allowed for emergency power shut off.

The above circuit design was implemented as a PCB using CAD software. Specifically, the stimulator circuitry layout was split across 4 modular PCBs to facilitate debugging: 1) mainboard which includes the MCUs, memory, storage, transceivers, 2) stimulation and charge balancing board which includes the circuit related to pulse generation, 3) multiplexer board, 4) touch-proof connector break-out board. The high-density flat flexible cables (FFC) are used to interconnect between the boards.

Similar to the prior BCI prototype, all stimulation control and data transmission for the BD-BCI system was wireless. Specifically, the base station software was designed with graphical user interface (GUI) written in Visual C # that enabled full control of stimulation parameters including pulse widths, pulse frequency, train duration, current level, and channel selection. All commands were transmitted wirelessly via the TRX.

It is well known that somatosensory percepts and motor responses (e.g. muscle contraction) can be elicited with electrical stimulation of the primary sensory (S1) and motor cortices (M1), respectively. Furthermore, adjusting stimulation parameters such as current amplitude, pulse frequency, and pulse width can change the type or quality of sensation and provide a sense of ownership of an artificial limb. Therefore, the BD-BCI stimulator was designed to have full control over pulse frequency, pulse duration, train duration, and current level so as to potentially deliver a wide variety of evoked sensory percepts. The BD-BCI stimulator was designed to match or exceed the specifications of a commercial FDA approved stimulator (Natus Nicolet Cortical Stimulator, Natus Medical Inc.®, Pleasanton, CA; henceforth referred to as the commercial stimulator).

Benchtop validation was performed to determine whether the BD-BCI stimulator accurately delivers stimulation parameters as commanded. Its accuracy was compared to that of commercial stimulators to establish equivalence. To this end, the temporal responses to identical sets of commands will be compared and plotted to verify that the current/voltage level, pulse width scale correctly with the parameter sweeping across a 1 kΩ resistive load. The integrity of the signals was verified by time-aligning and overlaying the waveforms over a set period of time (e.g. 5 seconds) per sweeping current and pulse widths. The accuracy of current level, pulse frequency, pulse width, and train duration was assessed by comparing the user-requested command versus measured values between the two stimulators.

The BD-BCI stimulator system was designed to conform to a stimulation charge density limit of 30 μC/cm² which was identified in previous animal studies as a safe limit. This safe limit was used in the first deep brain stimulator (DBS) approved in the US, the Medtronic Activa Tremor Control System (US FDA 1997) for essential tremor, as well as many other neural stimulator medical devices. The charge density (CD), expressed in μC/cm², is defined as: CD=I×PW/EA, where I: current (mA), PW: pulse width (ms), EA: exposed surface area of the electrode (cm²). CD was automatically calculated from the stimulation parameters commanded by the user via the GUI. As a protective measure, if the requested stimulation parameters exceed the 30 μcm² limit, the stimulator, and GUI enter into a “lock” mode until the parameters are altered to safe levels.

To detect if electrode-brain contact for any stimulation channel is adequate, the load impedance between the two stimulating electrodes was derived as follows. A short test stimulation pulse was delivered across the electrode pair. The voltage across the electrode pair was measured by the microcontroller's onboard ADC. Similarly, a current sense resistor/amplifier (Texas Instrument®, Dallas, TX) was used to measure the current. The impedance is then derived using Ohm's law. The stimulator was configured to “lockout” any channel with impedance >3 kΩ, indicating bad electrode-brain contact.

Impedance measurement was validated by comparing the true impedance (measured by a commercial digital multimeter, Kaiweets®, HT206D) versus the measured impedance (determined by the BD-BCI prototype) of the standard commercial through-hole resistors. Here, 8 different values of resistors between 330 Ω and 1,500 Ω were measured five times each. A regression analysis between the true and measured impedance was used to assess the validity of the BD-BCI prototype.

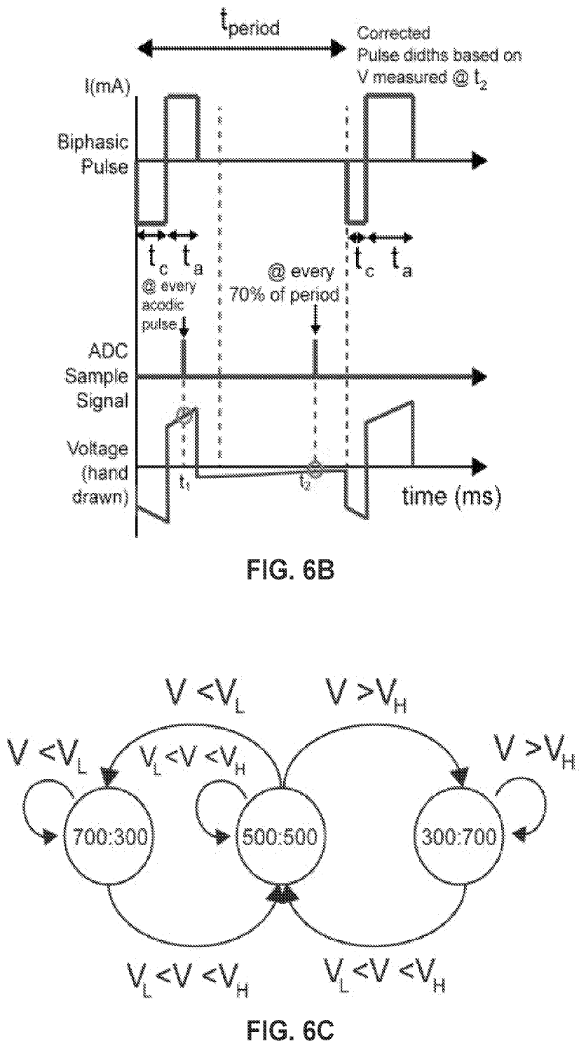

Passive and active charge balancing methods were used in the BD-BCI. The passive charge balancing was implemented by switching the H-bridge outputs at every off-duty cycle to GND to release residual charge accumulated at the stimulating electrodes. Active charge balancing utilized adjustments in stimulation pulse width to correct for any detected charge accumulation. Specifically, to measure the steady-state level potential at an electrode (given that residual voltage is directly related to charge), the voltage was sampled at 70% of every duty cycle between the pulses (FIG. 6B). This was achieved using a buffer amplifier (Texas Instrument®, Dallas, TX) and instrumentation amplifier (Texas Instrument®, Dallas, TX) cascaded as in FIG. 5. Based on the measured voltage (VMEAS), the microcontroller applied a corrective pulse. A state-machine (FIG. 6C) dictated how the ratio between cathodic and anodic pulse widths are correctively adjusted (e.g. 700:300 μs, respectively) at the very next pulse cycle from the cycle of measurement (FIG. 6B). According to this algorithm, when measured voltages exceed an arbitrary threshold, corrective pulses were generated to reverse the effect of the biased parameters to bring back the steady-state voltage below the thresholds.

To validate the charge balancing mechanism, 0 mV and −60 mV were set as the upper and lower tolerance thresholds (FIGS. 6A-6C). The system was tested to determine if the voltage (and thereby the charge) ever violates these thresholds. To this end, an anodic voltage offset was introduced to be corrected later with the corrective pulses (700:300 μs) according to the active charge balancing. The current and frequency were set to 12 mA and 200 Hz, respectively, and the stimulation was delivered continuously. With a bioamplifier (MP150, Biopac System, Inc.® Goleta, CA), the time-response of corrective pulses and the voltage at multiple neighboring electrodes in the ECoG grid were recorded when the active charge balancing function turned on and off over 20 seconds. The number of times that the voltage violated the upper or lower thresholds was determined.

The charge balancing and artifact mitigation tests were performed on a phantom brain tissue so as to mimic the environment for implanted ECoG electrodes to test the stimulator and its responses. The phantom brain tissue was created by mixing 6 g of food-grade agar powder into 100 ml of warm water (85-90° C.) with 50 mg of table salt. The solution was poured into a Petri dish and allowed to cool in a 4° C. refrigerator overnight. During testing, the phantom was warmed to room temperature, and a standard 8×8 ECoG grid (Ad-Tech®, Oak Creek, WI) with platinum electrodes (4 mm diameter, 2.3 mm Artifact mitigation strategies have been proposed including front-end techniques that focus on preventing saturation and rapid recovery in the amplifier and back-end techniques to recover the neural signals. By reducing the stimulation artifact in the nearby channels, saturation of amplifiers in the nearby brain area (e.g. on the motor cortex) can be prevented and the charge accumulation in the nearby electrodes can be further reduced. To reduce the impact of stimulation artifacts, an extra pole is added to the stimulating dipole which can be optionally activated to split the current into the extra pole to reduce the stimulation artifact amplitude. The extra pole can be selected with a multiplexer (A3 in FIG. 5) in a formation where two connected poles surround the other pole as an electromagnetic trap as in FIG. 5.

For assessment, the degree to which the artifact propagates to the nearby channels was verified by recording the time series data during stimulation with and without the artifact mitigation function. The percentage of artifact reduction was assessed by comparing the mean peak voltage of the artifacts before and after the method is applied.

After establishing the validity of the basic functions of the BD-BCI stimulator, it is necessary to determine that it can elicit similar behavioral responses as the commercial stimulator. The commercial stimulator was clinically used for functional brain mapping as part of the epilepsy surgery evaluation and acted as the benchmark target for the BD-BCI stimulator. The BD-BCI stimulator acquired an abbreviated investigational device exemption (IDE) and IRB approval at Rancho Los Amigos National Rehabilitation Center (Downey, CA). Patients undergoing intractable epilepsy surgery evaluation with ECoG electrode implantation over the left M1/S1 area were recruited for this experiment. Functional brain mapping with the commercial system was performed after anti-epileptic drugs (AEDs) were restarted to identify eloquent brain areas so that they can be spared from resection. After the clinical functional brain mapping was completed, the procedure was repeated with the BD-BCI stimulator to assess the equivalency of the patient's sensorimotor responses.

To this end, both the commercial and BD-BCI stimulator delivered bipolar stimulation with fixed frequency and pulse width, namely 50 Hz, 250 μs, respectively. The commercial system delivered 4 s pulse trains, whereas it was 2 s for the BD-BCI stimulator, as this shorter duration was sufficient to elicit sensorimotor percepts. To test for responses, stimulation was delivered starting at a minimum of 8 mA for the commercial stimulator and 3 mA for the BD-BCI stimulator. The current was increased incrementally by 2 mA for the commercial stimulator and by 1 mA for the BD-BCI stimulator, either until the patient reported a response or sensorimotor response was observed, or after discharge activity on ECoG prevented further current increase, or the current was high enough to establish no clinical response existed at that particular channel. If the BD-BCI stimulator's maximum available current for the particular channel is lower than the current that meets any of the above conditions, the stimulation was stopped at that current.

After each stimulation delivery, the patient was asked to report the perceived intensity, quality, and location of sensation or movement on the patient's body (the anatomical location was identified using a body map that divided the body surface into 45 compartments. The in vivo stimulation pulses delivered to the brain were measured and qualitatively compared between the commercial and BD-BCI stimulators using a commercial handheld oscilloscope (Siglent Technology®, Shenzhen, China) connected in parallel to a stimulating electrode pair. The equivalency between the two stimulators will be quantified based on the percentage of matching anatomical localization of the responses between the two stimulators.

The BD-BCI prototype PCBs were fabricated (Smart Prototyping®, Shenzhen, China) and assembled. The system weight is 164 g, and the case's dimensions are 7×9×5 cm (similar in size to a Raspberry Pi®). The GUI was implemented in C # as an add-on to the previous BCI GUI, which was executed on a desktop computer (Windows® 10), and facilitates control of all stimulation parameters and features via the base station. The BD-BCI prototype system software pertaining to stimulation was implemented in CH as an add-on to the previous BCI operating system, compiled, and deployed to the MCU cores of the multi-core MCU cluster.

Electrical stimulation was delivered to a pair of electrodes across a resistor by sweeping across current, pulse width, and frequency. The accuracy of the BD-BCI stimulator was compared with that of the commercial stimulator. There were high correlations between commanded and measured current for both the commercial (R²>0.99, intercept=−0.18, slope=1.03) and BD-BCI stimulator (R²>0.99, intercept=−0.11, slope=1.01), between commanded and measured pulse frequency for both the commercial (R²>0.99, intercept=−1.04, slope=1.00) and BD-BCI stimulator (R²>0.99, intercept=0.04, slope=1.00), between commanded and measured pulse width for both the commercial (R²>0.99, intercept=−1.14, slope=1.00) and BD-BCI stimulator (R²>0.99, intercept=0.28, slope=1.00), and between commanded and measured train duration for both the commercial (R²>0.99, intercept=0.00, slope=1.00) and BD-BCI stimulator (R²>0.99, intercept=0.00, slope=1.00). These results demonstrate that the BD-BCI stimulator has highly accurate control of stimulation parameters and was output-equivalent to the commercial stimulator in controlling the current level, pulse frequency, pulse width, and train duration.

Passive charge balancing ensured that there was no charge accumulation in the stimulation channels (E38, E39) although charge accumulation was seen in neighboring electrodes. The addition of an active charge balancing mechanism maintained the steady-state voltage between the upper and lower thresholds 100% of the time in neighboring electrodes in the test case (0 out of 400 k samples over the 20 s test period.

The mean peak voltage of the stimulation artifact decreased by 64.5% in E37, 31.8% in E23, 43.1% in E15 and 44.1% in E7, providing case evidence that the technique can be effective not only to the nearest neighbors to the stimulation dipole but throughout the distant measurement sights.

The R² between true and measured impedance was 0.996 (intercept=28.96, slope=0.962). An R² and slope near 1.0 indicates that there were strong linear associations between the true and measured impedance. The prospective charge density was automatically calculated from the requested stimulation parameters, and the GUI successfully “locks” if it is >30 μC/cm².

A single epilepsy patient (female, age 42) undergoing epilepsy surgery evaluation provided informed consent to participate in this experiment. The ECoG grid locations were identified by MRI-CT image fusion (electrode placement dictated by clinical needs). The mapping procedures with the BD-BCI stimulator were performed the day after clinical mapping was conducted with the commercial stimulator. Given the patient's limited availability, only half of the grid space was mapped with the BD-BCI stimulator, including electrodes in rows starting with electrodes 1, 2, 5, and 6. Bipolar stimulation with electrode pairs in vertical axes (e.g. 1-2, 9-10, 17-18, . . . ) was performed.

In the comparison of the patient's response to the functional brain mapping by the commercial and BD-BCI stimulator, all responses were motor, and neither system was able to elicit any patient response in electrodes overlying S1. In 2 channels, 5-6 and 29-30, the current parameters used to elicit a response with the commercial stimulator (>8 mA) were not available in the BD-BCI stimulator due to higher impedance (>1.7 kΩ) since the BD-BCI stimulator has lower output voltage (13V) compared to the commercial stimulator (24V), all of the matching grid space elicited response to the equivalent body location. 13 out of 13 electrode pairs (100%) produced responses to the same body location. The perceived intensity of behavioral responses could not be quantified and compared between systems as clinicians did not have adequate time to ask the patient during clinical mapping. Throughout experimentation, the patient received no additional AED, was seizure-free with no after-discharges or epileptiform discharges observed on ECoG signals, and expressed no subjective complaints.

The results show that the custom BD-BCI direct cortical stimulator has highly accurate parameter control and was comparable to that of an FDA-approved commercial stimulator. Additional safety features in the BD-BCI stimulator made it suitable for potential future chronic use. Furthermore, the BD-BCI stimulator was able to elicit identical sensorimotor responses to those of the commercial system in bedside testing. These aspects will be discussed in further detail below.

Both benchtop and bedside tests comparing the FDA-approved commercial stimulator and the BD-BCI stimulator revealed extremely high concordance between the accuracy of stimulation parameter control and the elicited sensorimotor responses. Therefore, it was concluded that the BD-BCI stimulator is output-equivalent to the FDA-approved commercial cortical stimulator in output and user-controllability across all features (bi-phasic current pulse, user-configurable current level, user-configurable pulse frequency, user-configurable pulse width, user-configurable train duration, activation stimulation indicator light, current output (mA), pulse frequency (Hz), pulse width (μs), train duration(s), max stimulation charge (μC), impedance monitoring, artifact monitoring, charge-monitoring/balancing, charge density monitoring (μC/ph/cm²). The equivalence to an FDA-approved stimulator provides a fully programmable and miniaturized stimulator architecture that can be used in future human studies in BD-BCI applications or other closed-loop neural stimulation studies and readily gain regulatory approval. Furthermore, it eliminates reliance on off-the-shelf systems with limited accessibility and portability, as seen in previously reported BD-BCI systems.

The combination of passive and active charge balancing was highly effective in removing voltage offsets (and thereby charge accumulation) that could develop in the stimulation electrodes and neighboring electrodes. The correction mechanism did not require more complex control algorithms such as PID control because the nature of rate of change of voltage is predictably slow which obviates the need for a derivative control. Similarly, because the target setpoint is a range rather than a point, the integration of error can be trivialized. The simple threshold-based active charge balancing was sufficient and minimized computational load to reserve processing power for more computationally demanding processes, e.g. decoding. Minimizing charge accumulation prevents damage to brain tissue and electrodes and thereby makes the BD-BCI stimulator design suitable for prospective use in long-term implantation scenarios. Considering that the advanced commercial deep brain stimulators are equipped only with passive charge-balancing mechanisms, and the existing BD-BCI systems are not integrated with advanced safety features, the combination of active and passive charge balancing in the fully-implantable brain stimulator system improves the BD-BCI prototype's future suitability for safe long-term stimulation.

With the current artifact mitigation technique, the mean peak voltage of the stimulation artifact reduction ranged from 31.8 to 64.5%. This front-end artifact mitigation technique reduces the risk of amplifier saturation, which is important to facilitate proper BD-BCI function. Specifically, decoding of M1 signals needs to occur simultaneously to S1 sensory stimulation, and such artifacts may confound or disrupt proper decoding. Although complete elimination of artifacts was not achieved here, mitigation of the artifact magnitude will make it easier for future back-end methods to remove the artifact and thereby achieve M1 decoding that is not confounded or disrupted by artifacts.

Even if stimulation is long-term charge-balanced, electrochemistry at the electrode-tissue interface may cause damage to the electrode and the tissue if an electrode is polarized during a stimulus pulse to an extent of causing irreversible redox reactions which could in turn electrolyze water, causing pH changes and gas formation, and electrode degradation. To mitigate the risk of these adverse reactions, a CD limit of 30 μcm² was used, which has been empirically characterized as safe in previous animal studies. CD monitoring and highly accurate impedance monitoring together ensure the safe delivery of the intended charge.

The work presented here is an especially important milestone as the combination of a fully programmable stimulator and decoder in a single embedded system provides all necessary hardware tools to design custom BD-BCIs for a variety of applications. For instance, in the example provided in the introduction, the BD-BCI can be applied to provide spatiotemporal information about one's limb during BCI-controlled movement, which may greatly reduce the risk of falling, dropping, etc. Although the BD-BCI prototype is roughly the size of a Raspberry Pi®, this bulk is difficult to translate into an implantable system. Further optimization with more advanced PCB design and smaller discrete components could drastically reduce the system footprint. Furthermore, translating the schematic into a custom IC may further reduce the footprint. Prior IC work has not reached a point where equivalency to FDA-approved commercial stimulators can be tested at benchtop and bedside. The system even be applied in non-BCI applications such as restoring somatosensory percepts to assist the planning and guiding of movements for persons with or without amputation or other forms of paralysis.

The fully programmable BD-BCI stimulator designed here is output-equivalent to the FDA-approved commercial stimulator and possesses additional safety features and functions that can facilitate chronic use. To date, there are no miniaturized and fully programmable cortical stimulators with such safety profiles. Achieving equivalency to an FDA-approved commercial stimulator enables future work to focus on further miniaturization and clinical applications in true BD-BCI operation, i.e. real-time brain-control of prosthetic limbs with simultaneous artificial sensory feedback.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A fully-implantable brain-computer interface system (100) comprising:
a. a skull unit (110), configured to be implanted in a skull of a patient;
b. a motor grid (120), comprising electrocorticogram (ECoG) motor electrodes, configured to be implanted in a subdural space over a motor area of a brain of the patient and configured to detect a brain wave motor signal, the motor grid (120) connected to the skull unit (110) via a wire;
c. a sensory grid (130), comprising ECoG sensory electrodes, configured to be implanted in a subdural space over a sensory area of the brain of the patient and configured to provide an electrical stimulation to the sensory area, the sensory grid (130) connected to the skull unit (110) via a wire;
d. a chest wall unit (140) configured to have wireless communication capabilities and configured to be implanted within a chest wall of the patient;
e. a subcutaneous tunneling cable (150) configured to connect the skull unit (110) and the chest wall unit (140); and
f. an external prosthetic system (160) comprising a sensor, configured for wireless communication with the chest wall unit (140);
wherein the system (100) is configured to control the external prosthetic system (160) based on the brain wave motor signal, and
wherein the system (100) is configured to provide the electrical stimulation to the sensory area based on activation of the sensor, thereby providing an artificial sensation to the patient.

2. The system (100) of claim 1, wherein the external prosthetic system (160) comprises a robotic gait exoskeleton (RGE), upper extremity prosthesis, upper extremity robotic exoskeleton, functional electrical stimulation system of upper or lower extremities, or spinal cord stimulator for the upper or lower extremities.

3. The system (100) of claim 1, wherein the motor area of the brain of the patient comprises a left primary motor cortex limb area and a right primary motor cortex limb area.

4. The system (100) of claim 1, wherein the sensory area of the brain comprises a left primary sensory cortex limb area and a right primary motor cortex limb area.

5. The system (100) of claim 1, wherein the skull unit (110) comprises an amplifier area, an analog-to-digital converter, a stimulator multiplexor array, or a combination thereof.

6. The system (100) of claim 1, wherein the chest wall unit (140) comprises a multi-core microcontroller (MCU) cluster, a memory and storage component, and a radio transceiver.

7. The system (100) of claim 1 further comprising a rechargeable battery configured to recharge wirelessly.

8. A fully implantable brain-computer interface (BCI) to allow for simultaneous control of a body part of a patient and sensation from the body part, the BCI comprising:
a. a skull unit (110) configured to be implanted within a skull of the patient;
b. a motor grid (120), comprising electrocorticogram (ECoG) motor electrodes, configured to be implanted in a subdural space over a motor area of a brain of the patient and configured to detect a brain wave motor signal, the motor grid (120) connected to the skull unit (110) via a wire; and
c. a sensory grid (130), comprising ECoG sensory electrodes, configured to be implanted in a subdural space over a sensory area of the brain and configured to provide an electrical stimulation to the sensory area, the sensory grid (130) connected to the skull unit (110) via a wire;
wherein the skull unit (110) is configured to allow for control of the body part using the brain wave motor signal and sensation from the body part using the electrical stimulation to the sensory area.

9. The BCI of claim 8, wherein the motor area of the brain of the patient comprises a left primary motor cortex limb area and a right primary motor cortex limb area.

10. The BCI of claim 8, wherein the sensory area of the brain comprises a left primary sensory cortex limb area and a right primary motor cortex limb area.

11. The BCI of claim 8, wherein the skull unit (110) comprises an amplifier area, an analog-to-digital converter, a stimulator multiplexor array, or a combination thereof.

12. The BCI of claim 8 further comprising a chest wall unit (140) comprising a multi-core microcontroller (MCU) cluster, a memory and storage component, and a radio transceiver, configured to have communication capabilities with the skull unit (110) and configured to be implanted within a chest wall of the patient.

13. The BCI of claim 8 further comprising a rechargeable battery configured to recharge wirelessly.

14.-20. (canceled)

21. A fully-implantable brain-computer interface system (100) comprising:
a. a motor grid (120), comprising electrocorticogram (ECoG) motor electrodes, configured to detect a brain wave motor signal;
b. a first unit (110) operatively connected to the motor grid (120) via a first wire, wherein the first unit (110) is configured to amplify and serialize the brain wave motor signal into a single path;
c. a sensory grid (130), comprising ECoG sensory electrodes, configured to provide an electrical stimulation, wherein the sensory grid (130) is operatively connected to the first unit (110) via a second wire;
d. a second unit (140) configured to have wireless communication capabilities;
e. a cable (150) configured to connect the first unit (110) and the second unit (140); and
f. an external prosthetic system (160) comprising a sensor, configured for wireless communication with the second unit (140);
wherein the system (100) is configured to control the external prosthetic system (160) based on the brain wave motor signal, and
wherein the system (100) is configured to provide the electrical stimulation based on activation of the sensor.

22. The system (100) of claim 21, wherein the external prosthetic system (160) comprises a robotic gait exoskeleton (RGE), upper extremity prosthesis, upper extremity robotic exoskeleton, or functional electrical stimulation system.

23. The system (100) of claim 21, wherein the first unit (110) comprises an amplifier area, an analog-to-digital converter, a stimulator multiplexor array, or a combination thereof.

24. The system (100) of claim 21, wherein the second unit (140) comprises a multi-core microcontroller (MCU) cluster, a memory and storage component, and a radio transceiver.

25. The system (100) of claim 21 further comprising a rechargeable battery configured to recharge wirelessly.
